(19) United States
(12) Patent Application Publication
(10) Pub. No.: US 2023/0187032 A1
(43) Pub. Date: Jun. 15, 2023

(54) PARENT-AND-CHILD HEALTH STUDY ASSISTANCE SYSTEM, PARENT-AND-CHILD HEALTH STUDY ASSISTANCE APPARATUS, AND DATA PROCESSING METHOD

(71) Applicants: NATIONAL UNIVERSITY CORPORATION HOKKAIDO UNIVERSITY, Sapporo-shi, Hokkaido (JP); MORINAGA MILK INDUSTRY CO., LTD., Tokyo (JP); Hitachi, Ltd., Tokyo (JP)

(72) Inventors: Daisuke SUZUKI, Tokyo (JP); Miaomei LEI, Tokyo (JP); Akiko TAMAKOSHI, Sapporo-shi, Hokkaido (JP); Takashi YOKOTA, Sapporo-shi, Hokkaido (JP); Kiminori NAKAMURA, Sapporo-shi, Hokkaido (JP); Yasuhiro TAKEDA, Kanagawa (JP); Tatsuya EHARA, Kanagawa (JP)

(21) Appl. No.: 17/917,056

(22) PCT Filed: Mar. 12, 2021
(86) PCT No.: PCT/JP2021/010013

§ 371 Date: Oct. 5, 2022

(30) Foreign Application Priority Data
Jun. 19, 2020 (JP) 2020-106030

(51) Int. Cl. G16H 10/20; G06Q 30/0208; G16H 20/60; G06F 21/62

(52) U.S. Cl. CPC G16H 10/20; G06Q 30/0208; G16H 20/60; G06F 21/6245

(57) ABSTRACT

A parent-and-child health study assistance system includes a data management section managing health study data used for a health study of at least one of a parent and a child in a family including a user as a family member, a level calculation section calculating a contribution level for the health study of the user based on input of the health study data, an incentive provision section providing the user with an incentive used as a profit corresponding to the contribution level, in conjunction with input of the health study data, an access control section managing an access authority to access the health study data, and an analysis result provision section providing an analysis result of analysis processing using the health study data.

### INCORPORATION BY REFERENCE

The present application claims the priority to Japanese Patent Application No. 2020-106030 filed on Jun. 19, 2020, the contents of which are incorporated herein by reference.

### TECHNICAL FIELD

The present invention relates to a technology for assisting parent-and-child health studies.

### BACKGROUND ART

For the purpose of assisting healthy development of children, and the like, cohort studies have started to be conducted in which lifestyle habits and physical conditions of mothers and children are studied during pregnancy and after childbirth. In systems utilized, for example, for cohort studies on the causes and effects of diseases and the like, advice for improvement of the life is presented to users in parallel with studies, analysis, and the like (see, for example, Patent Document 1).

Patent Document 1 discloses that “a health information management system first registers registration information of a monitoring subject in a monitoring subject information storage section 151. The health information management system then sets a health level on the basis of the registration information, and stores, in a biological information storage section 153, biological information measured according to the health level and transmitted by a monitoring unit. Then, the health information management system executes abnormality determination processing for the monitoring subject on the basis of the biological information stored in the biological information storage section 153 and executes processing for presenting advice information to the monitoring subject according to the resultant abnormality determination.

### PRIOR ART DOCUMENT

### Patent Document

Patent Document 1: JP-2003-310560-A

### SUMMARY OF THE INVENTION

### Problem to be Solved by the Invention

For the purpose of assisting healthy development of children, systems are available that acquire and analyze the lifestyle habit and physical condition of each of a parent and a child. Known systems take much time from input of data such as answers to a questionnaire until feedback of an analysis result. Additionally, in the initial stage of system introduction, sufficient knowledge has not been acquired, and thus accurate feedback is difficult to provide.

Thus, users do not feel the advantage of use of the system, and this prevents incenting for use of the system from being enhanced. This may in turn lead to difficulty in promoting use by new users and continued use by existing users.

An object of the present invention is to implement a mechanism for providing users with the advantage which promote use of the parent-and-child health study system.

### Means for Solving the Problems

A representative example of the invention disclosed in the present application is disclosed below. Specifically, a parent-and-child health study assistance system including a plurality of terminals held by a user, and at least one computer connected to the plurality of terminals is provided, the at least one computer including an arithmetic apparatus and a storage apparatus connected to the arithmetic apparatus, the parent-and-child health study assistance system including a data management section managing health study data used for a health study of at least one of a parent and a child in a family including the user as a member, a level calculation section calculating a contribution level for the health study of the user based on input of the health study data, an incentive provision section providing a first incentive used as a profit corresponding to the contribution level, to the user via the terminal in conjunction with input of the health study data, an access control section managing an access authority to access the health study data, and an analysis result provision section providing an analysis result of analysis processing using the health study data, the access control section determining, on a basis of an attribute of a provider providing a service, the access authority granted to the provider.

### Advantages of the Invention

According to the present invention, the user can enjoy the incentive (profit) involved in the use of the parent-and-child health study assistance system, allowing the use of the system to be promoted. Other objects, configurations, and effects will be clear from description of embodiments below.

### BRIEF DESCRIPTION OF THE DRAWINGS

FIG. 1 is a diagram depicting an example of a configuration of a whole system including a parent-and-child health study assistance system of Embodiment 1.

FIG. 2 is a diagram depicting an example of a hardware configuration of the parent-and-child health study assistance system of Embodiment 1.

FIG. 3 is a diagram depicting an example of a data structure of a collaborator management DB of Embodiment 1.

FIG. 4 is a diagram depicting an example of a data structure of a health study data management DB of Embodiment 1.

FIG. 5 is a diagram depicting an example of a data structure of a text management DB of Embodiment 1.

FIG. 6 is a diagram depicting an example of a data structure of a service provider management DB of Embodiment 1.

FIG. 7 is a diagram depicting an example of a data structure of a service management DB of Embodiment 1.

FIG. 8 is a diagram depicting an example of a data structure of an incentive management DB of Embodiment 1.

FIG. 9 is a diagram depicting an example of input screen presentation processing executed by the parent-and-child health study assistance system of Embodiment 1.

FIG. 10 is a diagram depicting an example of a data input screen displayed on a mobile terminal of Embodiment 1.

FIG. 11 is a flowchart illustrating an example of health study data reception processing executed by the parent-and-child health study assistance system of Embodiment 1.

FIG. 12 is a flowchart illustrating an example of text data reception processing executed by the parent-and-child health study assistance system of Embodiment 1.

FIG. 13 is a flowchart illustrating an example of service provider registration processing executed by the parent-and-child health study assistance system of Embodiment 1.

FIG. 14 is a diagram depicting an example of a registration screen presented to a system of Embodiment 1.

FIG. 15 is a flowchart illustrating an example of service registration processing executed by the parent-and-child health study assistance system of Embodiment 1.

FIG. 16 is a diagram depicting an example of a registration screen presented to the system of Embodiment 1.

FIG. 17 is a flowchart illustrating an example of health study data access control processing executed by the parent-and-child health study assistance system of Embodiment 1.

FIG. 18 is a flowchart illustrating an example of analysis result reception processing executed by the parent-and-child health study assistance system of Embodiment 1.

### MODES FOR CARRYING OUT THE INVENTION

Embodiments of the present invention will be described below using the drawings. However, the present invention is not intended to be interpreted by being limited to the description of the embodiments below. A person skilled in the art easily comprehends that the specific configuration of the present invention may be modified without departing from the concepts or spirits of the present invention. In the configuration of the invention described below, the same or similar components or functions are denoted by the same reference numerals, and duplicate descriptions are omitted. The notations “first,” “second,” “third,” and the like in the specification and the like are intended to identify components and are not necessarily intended to limit the number or order of the components. For facilitation of understanding of the invention, the positions, sizes, shapes, ranges, and the like of the components depicted in the drawings and the like may not represent the actual positions, sizes, shapes, ranges, and the like. Thus, the present invention is not limited to the positions, sizes, shapes, ranges, and the like of the components disclosed in the drawings and the like.

### Embodiment 1

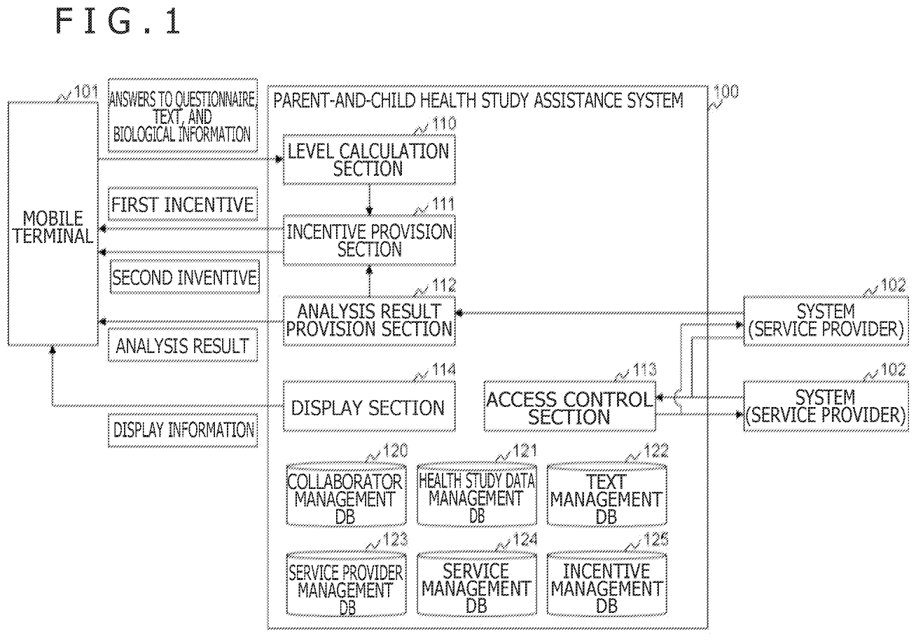

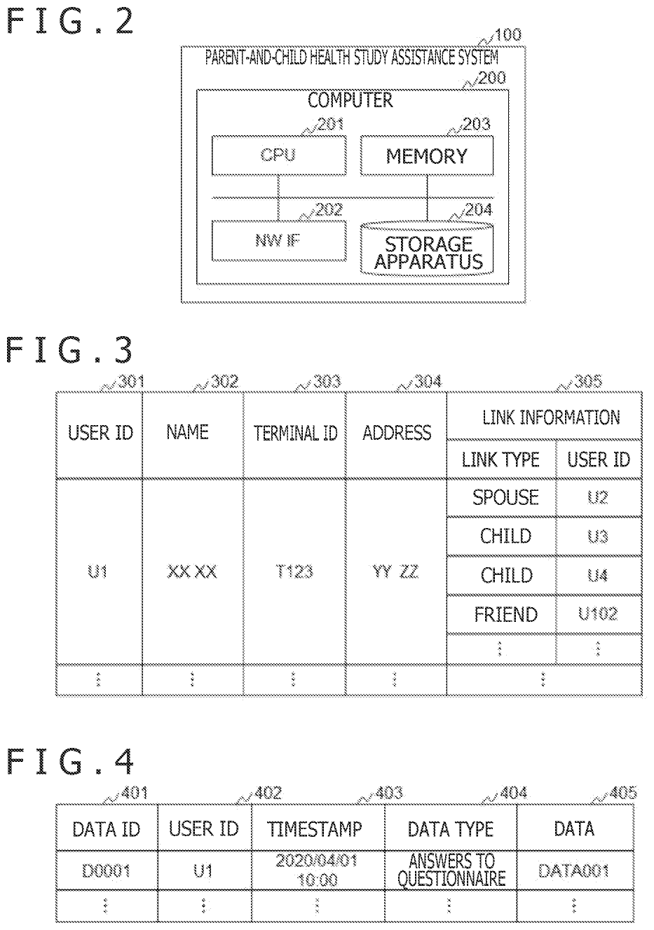

FIG. 1 is a diagram depicting an example of a configuration of a whole system including a parent-and-child health study assistance system of Embodiment 1. FIG. 2 is a diagram depicting an example of a hardware configuration of the parent-and-child health study assistance system of Embodiment 1.

A parent-and-child health study assistance system 100 is connected to a mobile terminal 101 used by a user. Additionally, the parent-and-child health study assistance system 100 is connected to a system 102 of a service provider providing a service. Note that in FIG. 1, one mobile terminal 101 is connected to the parent-and-child health study assistance system 100 but that, in actuality, a plurality of the mobile terminals 101 are connected to the parent-and-child health study assistance system.

The parent-and-child health study assistance system 100 and the mobile terminal 101 are connected to each other via a network such as a WAN (Wide Area Network) or a LAN (Local Area network). A connection scheme for the network may be either wired or wireless. Additionally, the parent-and-child health study assistance system 100 and the system 102 are connected to each other via a network.

Note that the parent-and-child health study assistance system 100 and the mobile terminal 101 may be directly connected together. Additionally, the parent-and-child health study assistance system 100 and the system 102 may be directly connected together.

The mobile terminal 101 is a terminal such as a smartphone. A user uses the mobile terminal 101 to input answers to a questionnaire, text, biological information, and the like. In the description below, answers to a questionnaire, text, and biological information for determining a physical condition of either a parent or a child are also described as health study data. Additionally, the user receives, via the mobile terminal 101, feedback to the input of the health study data, including an analysis result, advice, and the like.

Note that instead of the mobile terminal 101, a personal computer, a bidirectional television, or the like may be used.

The service provider includes a research institute, a local government, a general corporation, and the like. The research institute performs analysis using health study data, and provides an analysis result and advice based on the analysis result. The local government provides an administrative service using health study data. The general corporation provides a service such as provision of products or information useful for health improvement. Note that the research institute may provide a service similar to that provided by the general corporation or that the local government and the general corporation may perform analysis using the health study data.

The parent-and-child health study assistance system 100 accumulates and provides health study data, and provides an analysis result and advice. As depicted in FIG. 2, the parent-and-child health study assistance system 100 includes at least one computer 200.

The computer 200 includes a CPU 201, a network interface 202, a memory 203, and a storage apparatus 204. The pieces of hardware are connected together via a bus. Note that the computer 200 may include an input apparatus such as a keyboard, a mouse, and a touch panel, and an output apparatus such as a display.

The CPU 201 executes programs stored in the memory 203. The CPU 201 executes processing in accordance with the programs to operate as functional sections (modules) implementing specific functions. In the description below, in a case where processing is described using the functional section as a subject, this indicates that the CPU 201 is executing the program implementing the functional section.

The network interface 202 is an interface for communicating with an external apparatus via a network.

The memory 203 stores the programs executed by the CPU 201 and information used by the programs. The memory 203 is used as a work area temporarily used by the programs. The storage apparatus 204 permanently stores information. The storage apparatus 204 includes an HDD (Hard Disk Drive), an SSD (Solid State Drive), and the like.

The memory 203 stores programs implementing a level calculation section 110, an incentive provision section 111, an analysis result provision section 112, an access control section 113, and a display section 114. The storage apparatus 204 stored a collaborator management DB 120, a health study data management DB 121, a text management DB 122, a service provider management DB 123, a service management DB 124, and an incentive management DB 125.

The level calculation section 110 calculates an incentive level indicating the contribution level for the health study of the user based on input of health study data. The level calculation section 110 inputs the incentive level to the incentive provision section 111.

The analysis result provision section 112 provides the user with at least either the analysis result or advice. Additionally, the analysis result provision section 112 inputs the analysis result and the advice to the incentive provision section 111.

The incentive provision section 111 provides an incentive to the user. In this regard, the incentive is a profit incenting input of health study data, that is, participation in a health study. The analysis result and the advice are examples of the incentive.

In the present embodiment, the parent-and-child health study assistance system 100 provides incentives in a step-by-step manner aside from the analysis result and the advice. Specifically, the parent-and-child health study assistance system 100 provides a first incentive when the health study data is input, and provides a second incentive when the analysis result is received. This allows new participation in the health study and continued participation in the health study to be promoted.

The access control section 113 controls access to a database. Additionally, the access control section 113 sets an access authority to access the database on the basis of the type of the service provided by the service provider.

The display section 114 generates display information for displaying various pieces of information on the mobile terminal 101.

Note that the parent-and-child health study assistance system 100 holds authority conversion information and applicable key conversion information aside from the database described above. In the authority conversion information, records are stored with which the service type and the access authority are associated. In the applicable key conversion information, records are stored with which an application condition and an applicable key are associated. In this regard, the applicable key is a key designating an incentive to be provided.

Now, the database will be described in detail using FIGS. 3 to 8.

FIG. 3 is a diagram depicting an example of a data structure of the collaborator management DB 120 of Embodiment 1.

The collaborator management DB 120 stores information related to users (collaborators) participating in the health study. The collaborator management DB 120 stores, for example, a plurality of records depicted in FIG. 3. One record corresponds to one user.

The record includes a user ID 301, a name 302, a terminal ID 303, an address 304, and link information 305.

The user ID 301 is a field in which identification information for uniquely identifying the user is stored. The name 302 is a field in which the name of the user is stored. The terminal ID 303 is a field in which identification information of the mobile terminal 101 used by the user is stored. Note that in the terminal ID 303 in the record of a child, the identification information of the mobile terminal 101 used by the parent may be stored or that the terminal ID 303 may be left blank. The address 304 is a field in which the residential address of the user is stored.

The link information 305 is a field in which information regarding another user having some relationship with the user. In the link information 305, rows including the identification information (user IDs) of other users and the link types are stored. In this regard, the link type indicates the relationship between the users. For example, “spouse,” “parent,” “child,” or “community” is set as the link type. “Community” represents an acquaintance on the SNS (Social Networking Service). Of course, the information stored in the collaborator management DB 120 is acquired with the consent of the user and managed with attention paid to security and the like.

FIG. 4 is a diagram depicting an example of a data structure of the health study data management DB 121 of Embodiment 1.

The health study data management DB 121 stores health study data received by the parent-and-child health study assistance system 100. The health study data management DB 121 stores, for example, a plurality of records depicted in FIG. 4. One record corresponds to one health study data.

The record includes a data ID 401, a user ID 402, a timestamp 403, a data type 404, and data 405.

The data ID 401 is a field in which identification information for uniquely identifying the health study data is stored. The user ID 402 is a field in which the identification information of the user having input the health study data is stored. The timestamp 403 is a field in which the date and time when the health study data has been input are stored.

The data type 404 is a field in which the type of the health study data is stored. In the data type 404, “answers to questionnaire,” “biological information (feces),” “biological information (breast milk),” “biological information (blood)” and the like are stored. The data 405 is a field in which the health study data is stored.

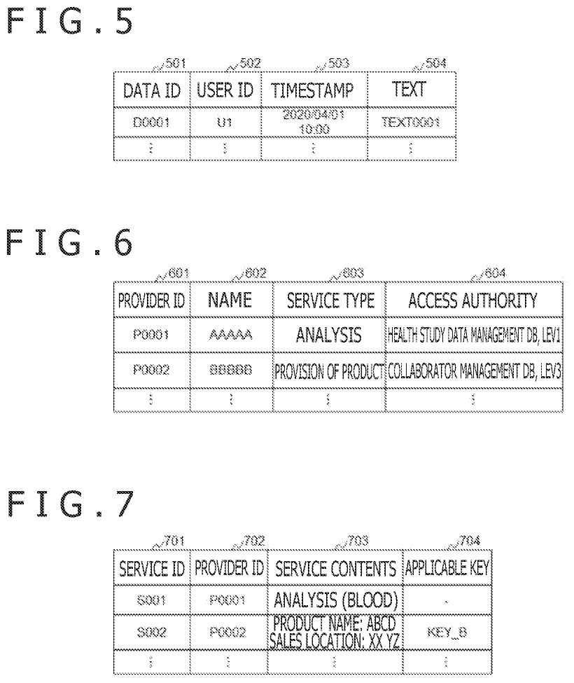

FIG. 5 is a diagram depicting an example of a data structure of the text management DB 122 of Embodiment 1.

The text management DB 122 stores a text input together with or separately from health study data. The text management DB 122 stores, for example, a plurality of records depicted in FIG. 5. One record corresponds to one text.

The record includes a data ID 501, a user ID 502, a timestamp 503, and a text 504.

The data ID 501 is a field in which identification information for uniquely identifying a text is stored. The user ID 502 is a field in which the identification information of the user having input the text is stored. The timestamp 503 is a field in which the date and time when the text has been input is stored. The text 504 is a field in which the text is stored.

FIG. 6 is a diagram depicting an example of a data structure of the service provider management DB 123 of Embodiment 1.

The service provider management DB 123 stores information related to the service provider. The service provider management DB 123 stores, for example, a plurality of the records. One record corresponds to one service provider. Note that the service provider may be replaced with a corporation.

The record includes a provider ID 601, a name 602, a service type 603, and an access authority 604.

The provider ID 601 is a field in which identification information for uniquely identifying the service provider is stored. The name 602 is a field in which the name of the service provider is stored. In the name 602, for example, the name of a research institute, a corporation, a local government, and a school is stored. The service provider 603 is a field in which the type of the service provided by the service provider is stored. In the service type 603, for example, “analysis,” “sale of products,” “provision of meals,” “provision of menu planning,” and “provision of meteorological information” are stored.

The access authority 604 is a field in which the access authority set for the service provider is stored. In the access authority 604, data is stored that corresponds to a combination of an accessible database and an access level. The access level indicates accessible items. The access level includes the level at which all items are accessible, the level at which items other than those which enable individuals to be identified are accessible, and the like.

Note that the link type is used to control access to the health study data or the like among the users. For example, access from a user with the link type “community” is controlled such that the user can access only the items other than those which enable individuals to be identified.

FIG. 7 is a diagram depicting an example of a data structure of the service management DB 124 of Embodiment 1.

The service management DB 124 stores information related to the service provided by the service provider. The service management DB 124 stores, for example, a plurality of records depicted in FIG. 7. One record corresponds to one service.

The record includes a service ID 701, a provider ID 702, a service content 703, and an applicable key 704.

The service ID 701 is a field in which identification information for uniquely identifying the service is stored. The provider ID 702 is a field in which the identification information of the service provider is stored. The service content 703 is a field in which the specific content of the service is stored. For example, the name of the product or the service, and the place of provision are stored. The applicable key 704 is a field in which the applicable key is stored. Note that a plurality of applicable keys may be associated with one service.

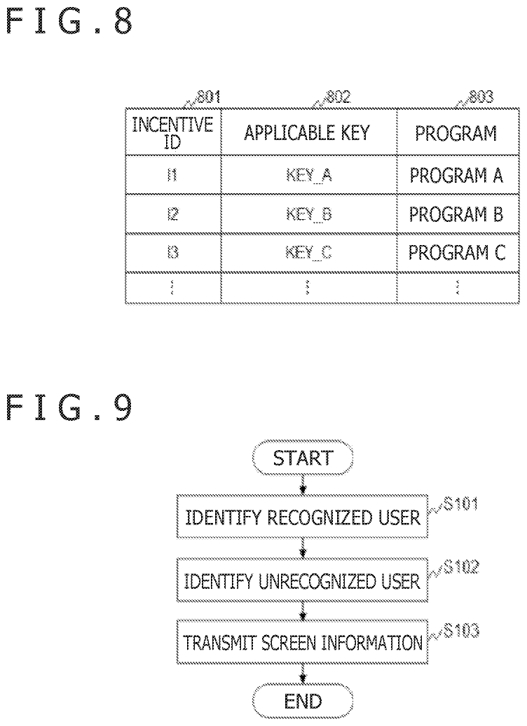

FIG. 8 is a diagram depicting an example of a data structure of the incentive management DB 125 of Embodiment 1.

The incentive management DB 125 stores information for controlling provision of an incentive. The incentive management DB 125 stores, for example, a plurality of records depicted in FIG. 8. One record corresponds to information for controlling provision of one incentive.

The record includes an incentive ID 801, an applicable key 802, and a program 803.

The incentive ID 801 is a field in which identification information for uniquely identifying an incentive to be provided is stored. The applicable key 802 is a field in which the applicable key is stored. The program 803 is a field in which programs for executing various processing operations for providing incentives are stored. In the present embodiment, programs are prepared according to the types of the incentives to be provided.

Now, processing executed by the parent-and-child health study assistance system 100 will be described.

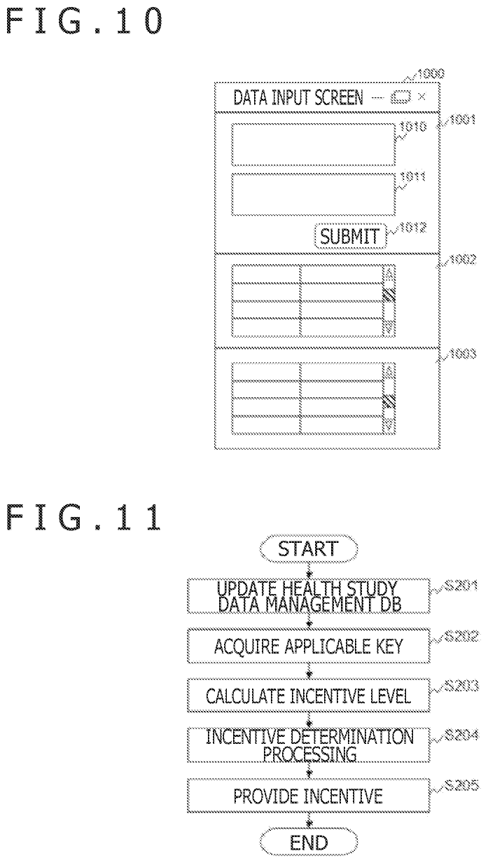

FIG. 9 is a diagram depicting an example of input screen presentation processing executed by the parent-and-child health study assistance system 100 of Embodiment 1. FIG. 10 is a diagram depicting an example of a data input screen displayed on the mobile terminal 101 of Embodiment 1.

First, a data input screen 1000 displayed on the mobile terminal 101 will be described using FIG. 10.

The data input screen 1000 includes a data input field 1001, a recognized user display field 1002, and an unrecognized user display field 1003.

The data input field 1001 is a field to which data to be transmitted to the parent-and-child health study assistance system 100 is input. The data input field 1001 includes a health study data field 1010, a text field 1011, and a submit button 1012. The health study data field 1010 is a field to which health study data is input. The text field 1011 is a field to which a pleasant event, the behavior of the user, a visited place, sympathy, an inconvenient matter, and the like are input as text. The submit button 1012 is an operation button for transmitting, to the parent-and-child health study assistance system 100, data input to one of the health study data field 1010 and the text field 1011.

The recognized user display field 1002 is a field displaying information regarding another user (recognized user) who can be directly contacted. The recognized user display field 1002 displays a table indicating the ID and the name of the user.

The unrecognized user display field 1003 is a field displaying information regarding another user who cannot be directly contacted at this stage and who has some relationship with the user. The unrecognized user display field 1003 displays a table indicating the ID and the name or nickname of the user.

Now, processing executed by the parent-and-child health study assistance system 100 to display the data input screen 1000 will be displayed using FIG. 9.

In a case of receiving, from the mobile terminal 101, a display request including a user ID, the parent-and-child health study assistance system 100 initiates input screen presentation processing.

The display section 114 identifies a recognized user on the basis of the collaborator management DB 120 (step S101). Specifically, processing as described below is executed.

The display section 114 outputs, to the access control section 113, a name acquisition request including a user ID.

The access control section 113 retrieves, from the collaborator management DB 120, a record including the user ID 301 in which the user ID included in the name acquisition request is set. The access control section 113 acquires user IDs in the respective rows of the link information 305 in the retrieved record. The access control section 113 retrieves a record including the user ID 301 in which the user ID acquired is set, and acquires the value of the name 302 in the retrieved record. The access control section 113 outputs, to the display section 114, data with which the user ID and the name are associated.

The display section 114 generates recognized user display information on the basis of data received from the access control section 113. The processing in step S101 has been described above.

The display section 114 identifies an unrecognized user on the basis of the text management DB 122 (step S102). Specifically, processing described below is executed.

The display section 114 outputs, to the access control section 113, a text acquisition request including a user ID.

The access control section 113 retrieves, from the text management DB 122, a record including the user ID 502 in which the user ID included in the text acquisition request is set. The access control section 113 reads out the text stored in the text 504 in the retrieved record, and outputs the read-out text to the display section 114. At this time, the access control section 113 may transmit only the text in the record included within a time range corresponding to a certain period of time before the current time.

The display section 114 performs text analysis on the read-out text and extracts matching keywords. Matching keywords for items such as time, place, person, and emotion are extracted. Well-known technologies may be used for the text analysis, and thus detailed description of the text analysis is omitted. The display section 114 outputs, to the access control section 113, a record retrieval request including the matching keywords.

The access control section 113 references the text 504 of each record in the text management DB 122 to retrieve records in which texts including the matching keywords are stored. At this time, the access control section 113 may retrieve a group of records included within a time range corresponding to a certain period of time before the current time. The access control section 113 outputs the retrieved records to the display section 114.

The display section 114 compiles the received records for each user, and uses the texts in the group of records for each user to calculate an association score for each user. For example, the display section 114 calculates the number of appearances of the matching keywords in the records of one user, and calculates the total value of the number of appearances as the association score. The display section 114 identifies, as an unrecognized user, a user with an association score larger than a threshold value. The display section 114 outputs, to the access control section 113, a name acquisition request including the ID of the unrecognized user.

The access control section 113 retrieves, from the collaborator management DB 120, a record including the user ID 301 in which the user ID included in the name acquisition request is set, and acquires the value of the name 302 in the retrieved record. The access control section 113 outputs, to the display section 114, data with which the user ID and the name are associated.

The display section 114 generates unrecognized user display information on the basis of data received from the access control section 113. Note that unrecognized user display information is generated allowing unrecognized users to be displayed in order of decreasing association score. The processing in step S102 has been described.

The display section 114 generates display information for displaying the health study data field 1010, compiles the display information, the recognized user display information, and the unrecognized user display information, and transmits the compiled information to the mobile terminal 101 as image information (step 103). Subsequently, the display section 114 ends the input screen presentation processing.

On the basis of the image information, the mobile terminal 101 displays, on the display, the data input screen 1000 depicted in FIG. 10.

The user references the unrecognized user display field 1003, contacts the unrecognized user, and registers the unrecognized user as a recognized user.

For example, the user displays a cord for participation on the display of the mobile terminal 101, and by using the camera of the mobile terminal 101 to load the cord, the unrecognized user transmits a recognized user registration request to the parent-and-child health study assistance system 100. Note that the cord varies depending on the link type. Additionally, the registration request includes the IDs of the user and the unrecognized user, and the link type corresponding to the cord. In this case, the access control section 113 retrieves, from the collaborator management DB 120, a record including the user ID 301 in which the user ID included in the registration request is set, and adds a row to the link information 305 in the retrieved record. The access control section 113 sets, in the added row, the link type included in the registration request, and also sets, in the added row, the ID of the unrecognized user included in the registration request.

Promoting the contact among the users as described above allows revitalization of regional activities to be expected. Additionally, providing the place of contact among the users allows new and continued use of the parent-and-child health study assistance system 100 to be promoted.

FIG. 11 is a flowchart illustrating an example of health study data reception processing executed by the parent-and-child health study assistance system 100 of Embodiment 1.

In a case of receiving health study data from the mobile terminal 101 along with the ID of the mobile terminal 101, the parent-and-child health study assistance system 100 initiates health study data reception processing.

The incentive provision section 111 stores the health study data in the health study data management DB 121 via the access control section 113 (step S201). Specifically, processing as described below is executed.

The incentive provision section 111 identifies the data type of health study data. For example, the data type is identified on the basis of one of the extension, data format, and contents of the health study data. The incentive provision section 111 outputs, to the access control section 113, the registration request including the ID of the mobile terminal 101, the health study data, and the data type.

The access control section 113 references the collaborator management DB 120 to retrieve a record including the terminal ID 303 in which the ID of the mobile terminal 101 included in the registration request is set. The access control section 113 acquires the value of the user ID 301 in the retrieved record.

The access control section 113 adds a record to the health study data management DB 121 and sets identification information in the data ID 401 in the added record. The access control section 113 sets the user ID acquired in the user ID 402 in the added record, sets, in the data type 404, the data type included in the registration request, and sets the health study data in the data 405. Additionally, the access control section 113 sets the current time in the timestamp 403 in the added record. Note that in a case where a timestamp is applied to the health study data, the timestamp is set in the timestamp 403.

The access control section 113 outputs the user ID to the incentive provision section 111 as a response. The processing in step 5202 has been described.

The incentive provision section 111 references applicable key conversion information to acquire the applicable key corresponding to reception of the health study data (step S202), and outputs, to the level calculation section 110, an incentive level calculation request including the user ID.

The level calculation section 110 calculates the incentive level of the user using the mobile terminal 101 having transmitted the health study data (step S203). For example, processing as described below is executed.

The level calculation section 110 outputs, to the access control section 113, a request for retrieval of the health study data including the user ID.

The access control section 113 references the health study data management DB 121 to retrieve records including the user ID 402 in which the user ID included in the retrieval request is set. The access control section 113 outputs the retrieved records to the level calculation section 110.

The level calculation section 110 calculates the incentive level on the basis of the records acquired from the access control section 113, and outputs the incentive level to the incentive provision section 111. For example, a calculation method described below is possible.

### (Calculation Method 1)

The level calculation section 110 calculates the incentive level on the basis of the number of records (the number of times health study data has been input). For example, the level calculation section 110 references conversion information including the number of records and the incentive level that are associated with each other, to convert the number of records received into the incentive level.

### (Calculation Method 2)

The level calculation section 110 analyzes the contents of the health study data and calculates scores on the basis of an analysis result. The level calculation section 110 calculates the total value of the scores of the health study data as an incentive level.

Note that a calculation method other than that described above may be used. For example, the level calculation section 110 may calculate the incentive level in consideration of the number of rows included in the link information 305. The processing in step 5203 has been described.

The incentive provision section 111 executes incentive determination processing (step S204). Specifically, processing as described below is executed.

The incentive provision section 111 references the incentive management DB 125 to retrieve a record including the applicable key 802 in which the applicable key acquired is set. The incentive provision section 111 outputs an activation instruction including the incentive level, to the program stored in the program 803 in the retrieved record.

The program having received the activation instruction selects the incentive to be provided on the basis of the incentive level. For example, a coupon, points, a voucher, or electronic money corresponding to the incentive level is selected as the incentive. The program outputs the selected incentive to the incentive provision section 111. The processing in step S204 has been described.

The incentive provision section 111 transmits the incentive to the mobile terminal 101 corresponding to the source of the health study data (step S205). Subsequently, the incentive provision section 111 ends the health study data reception processing.

The analysis of the health study data takes much time, and thus there is a time lag between the input of the health study data and the provision of an analysis result. The time lag is a cause of prevention of promotion of use by new users and continued use by existing users. The parent-and-child health study assistance system 100 of the present embodiment provides an incentive corresponding to the degree of previous contribution (incentive level) to the health study. This allows the use by new users and the continued use by existing users to be promoted.

Note that the incentive may be provided by the administrator of the parent-and-child health study assistance system 100 or an organization to which the administrator belongs or may be provided by the service provider.

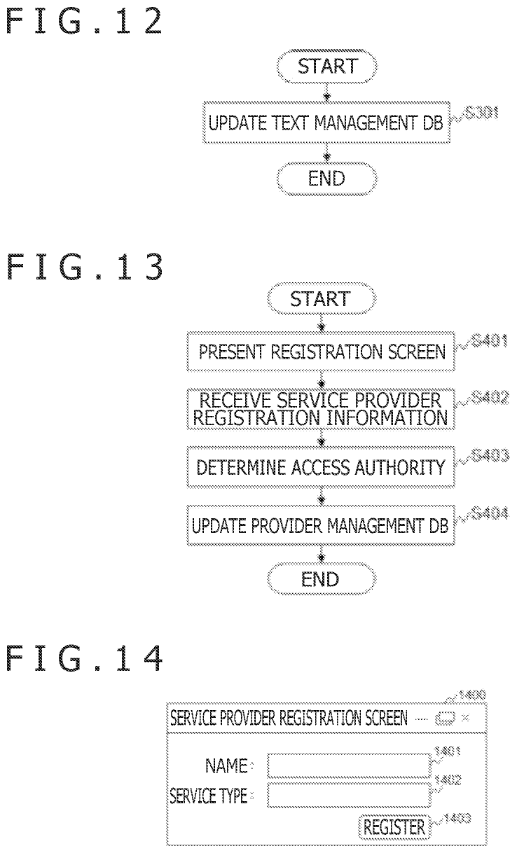

FIG. 12 is a flowchart illustrating an example of text data reception processing executed by the parent-and-child health study assistance system 100 of Embodiment 1.

In a case of receiving text data from the mobile terminal 101 along with the ID of the mobile terminal 101, the parent-and-child health study assistance system 100 initiates text data reception processing.

The access control section 113 stores the text data in the text management DB 122 (step S301). Subsequently, the access control section 113 ends the text data reception processing. Specifically, processing as described below is executed.

The access control section 113 references the collaborator management DB 120 to retrieve a record including the terminal ID 303 in which the received ID of the mobile terminal 101 is set. The access control section 113 acquires the value of the user ID 301 in the retrieved record.

The access control section 113 adds a record to the text management DB 122, and sets identification information in the data ID 501 in the added record. The access control section 113 sets the user ID acquired in the user ID 502 in the added record, and sets the received text data in the text 504. The access control section 113 sets the current time in the timestamp 503 in the added record. Note that in a case where a timestamp is applied to the text data, the timestamp is set in the timestamp 503.

FIG. 13 is a flowchart illustrating an example of service provider registration processing executed by the parent-and-child health study assistance system 100 of Embodiment 1. FIG. 14 is a diagram depicting an example of a registration screen presented to the system 102 of Embodiment 1.

In a case of receiving a registration request for a service provider from the system 102, the parent-and-child health study assistance system 100 initiates service provider registration processing.

The display section 114 presents a registration screen to the system 102 (step S401). A registration screen 1400 as depicted in FIG. 14 is presented to the system 102.

The registration screen 1400 includes a name field 1401, a service type field 1402, and a register button 1403. The name field 1401 is a field to which the name of the service provider is input. The service type field 1402 is a field to which the service type is input. The service provider may directly input a value to the service type field 1402 or may select a value from a pulldown menu. The register button 1403 is an operation button for transmitting service provider registration information.

The access control section 113 receives the service provider registration information from the system 102 (step S402).

The access control section 113 determines the access authority on the basis of the service type of the service provider (step S403).

Specifically, the access control section 113 references authority conversion information on the basis of the service type to determine the access authority of the service provider.

The access control section 113 updates the service provider management DB 123 on the basis of the service provider registration information and the determined access authority (step S404). Subsequently, the access control section 113 ends the service provider registration processing.

Specifically, the access control section 113 adds a record to the service provider management DB 123, and sets identification information in the provider ID 601 in the added record. The access control section 113 sets, in the name 602 and the service type 603 in the added record, values included in the service provider registration information. Additionally, the access control section 113 sets the determined access authority in the access authority 604 in the added record.

Note that the access control section 113 may notify the system 102 of the identification information set in the provider ID 601.

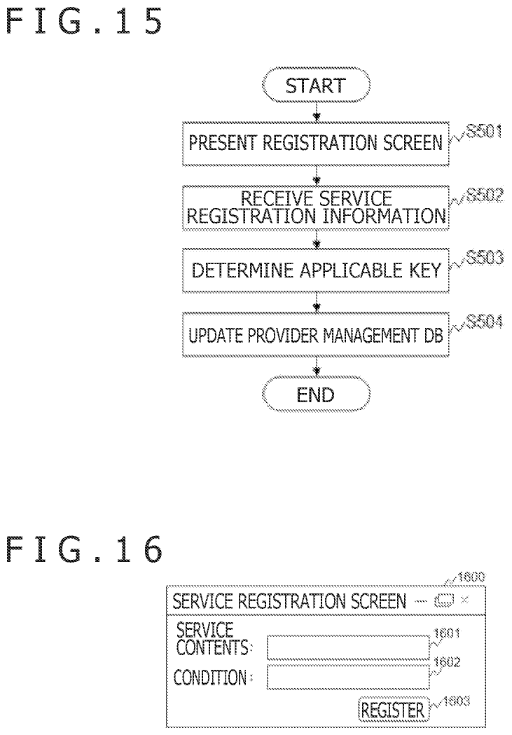

FIG. 15 is a flowchart illustrating an example of service registration processing executed by the parent-and-child health study assistance system 100 of Embodiment 1. FIG. 16 is a diagram depicting an example of a registration screen presented to the system 102 of Embodiment 1.

In a case of receiving a service registration request from the system 102, the parent-and-child health study assistance system 100 initiates service registration processing.

The display section 114 presents the registration screen to the system 102 (step S501). A registration screen 1600 as depicted in FIG. 16 is presented to the system 102.

The registration screen 1600 includes a service content field 1601, a condition field 1602, and a register button 1603. The service content field 1601 is a field to which the specific content of the service is input. The condition field 1602 is a field to which a service provision condition is input. In the present embodiment, the content of the analysis result is input as the service provision condition. For example, “deficiency of nutrient A” is input as the provision condition. Note that the service provider may directly input a value to the condition field 1602 or may select a value from a pulldown menu. The register button 1603 is an operation button for transmitting service registration information. The system 102 transmits the service registration information to the parent-and-child health study assistance system 100 along with the name or identification information of the service provider.

Note that in a case where the service content field 1601 indicates analysis, the condition field 1602 may be left blank.

The access control section 113 receives the service registration information from the system 102 (step S502).

At this time, the access control section 113 references the service provider management DB 123 to determine whether any service provider is registered. In a case where no service provider is registered in the service provider management DB 123, the access control section 113 notifies about an error.

The access control section 113 determines the applicable key on the basis of the provision condition included in the service registration information (step S503).

Specifically, the access control section 113 references the applicable key conversion information on the basis of the provision condition to determine the applicable key.

The access control section 113 updates the service management DB 124 on the basis of the service registration information and the determined applicable key (step S504). Subsequently, the access control section 113 ends the service registration processing.

Specifically, the access control section 113 adds a record to the service management DB 124, and sets identification information in the service ID 701 in the added record. The access control section 113 sets identification information of the service provider in the provider ID 702 in the added record, and sets, in the service content 703, a value included in the service registration information. Additionally, the access control section 113 sets the applicable key determined in the applicable key 704 in the added record.

The registration of the service to be provided as the second incentive has been described with reference to FIG. 15. However, the service to be provided as the first incentive can also registered. In this case, a program for executing various processing operations for providing the incentive is input to the system 102 as a service content. The input information is registered in the incentive management DB 125. Note that instead of the program, the service content and information indicating that the service is provided as the first incentive may be input. In this case, the access control section 113 provides notification to the administrator of the parent-and-child health study assistance system 100. The administrator modifies the program according to the notification and updates the incentive management DB 125.

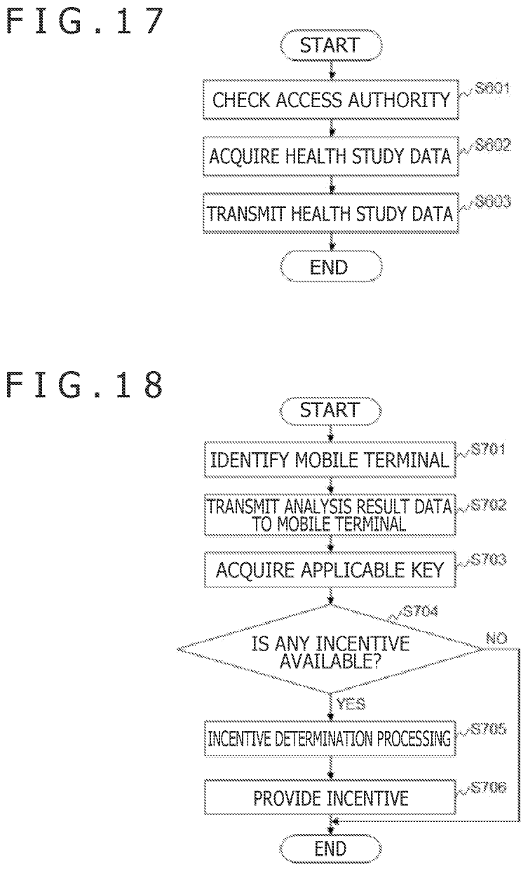

FIG. 17 is a flowchart illustrating an example of health study data access control processing executed by the parent-and-child health study assistance system 100 of Embodiment 1.

In a case of receiving health study data acquisition request from the system 102, the parent-and-child health study assistance system 100 initiates health study data access control processing. The health study data acquisition request includes the identification information of the service provider and an acquisition condition. The acquisition condition is a condition for specifying health study data to be acquired, and includes, for example, the time range, the identification information of the user, and the data type.

The access control section 113 references the service provider management DB 123 to check the access authority of the service provider (step S601). In a case where no access authority to access the health study data management DB 121 is granted to the service provider, the access control section 113 notifies the system 102 of an error. In this regard, it is assumed that the access authority to access the health study data management DB 121 is granted to the service provider.

On the basis of the access authority and the acquisition condition, the access control section 113 acquires health study data from the health study data management DB 121 (step S602).

At this time, the access control section 113 may perform a processing operation intended to make the health study data anonymous, and the like.

The access control section 113 transmits the health study data acquired to the system 102 corresponding to the source of the health study data acquisition request (step S603). Subsequently, the access control section 113 ends the health study data access control processing.

The system 102 uses the health study data acquired to execute analysis processing. For example, on the basis of the time when the user went out, the place where the user went out to, and the weather at the time when user went out, the system 102 calculates the time of sunbathing of the user, the exposure of ultraviolet radiation to the user, and the like, which can be determined from answers to questionnaires, and further, on the basis of the time of sunbathing of the user, the exposure of ultraviolet radiation to the user, and the like, calculates an estimated levels of vitamin D in the body of the user. Note that the place where the user went out to may be determined on the basis of GPS information received from the user. Additionally, the weather at the time when the user went out may be acquired from an external system providing information related to the weather.

FIG. 18 is a flowchart illustrating an example of analysis result reception processing executed by the parent-and-child health study assistance system 100 of Embodiment 1.

In a case of receiving analysis result data from the system 102, the parent-and-child health study assistance system 100 initiates analysis result reception processing. The analysis result data includes the identification information of the service provider, the user ID, and an analysis result.

The analysis result provision section 112 identifies the mobile terminal 101 transmitting analysis result data (step S701). Specifically, processing as described below is executed.

The analysis result provision section 112 outputs a terminal retrieval request including the user ID.

The access control section 113 references the collaborator management DB 120 to retrieve a record including the user ID 301 in which the user ID included in the terminal retrieval request is set. The access control section 113 reads out the terminal ID stored in the terminal ID 303 in the retrieved record, and outputs the terminal ID to the analysis result provision section 112. The processing in step S701 has been described.

The analysis result provision section 112 transmits analysis result data to the identified mobile terminal 101 (step S702). Additionally, the analysis result provision section 112 outputs the analysis result data to the incentive provision section 111.

The incentive provision section 111 references the applicable key conversion information to acquire an applicable key corresponding to the content of the analysis result included in the analysis result data (step S703).

By analyzing numerical values or character strings included in the analysis result, the incentive provision section 111 identifies the content of the analysis result. For example, in a case where the analysis result includes the intake of a nutrient A and an alert indicating the deficiency of the nutrient A, the incentive provision section 111 outputs “deficiency of nutrient A” as the content of the analysis result.

On the basis of the content of the analysis result, the incentive provision section 111 determines whether an available incentive is present (step S704). Specifically, processing as described below is executed.

The incentive provision section 111 outputs a service retrieval request including the applicable key to the access control section 113.

The access control section 113 references the service management DB 124 to retrieve a record including the applicable key 704 in which the applicable key included in the service retrieval request is stored. The access control section 113 outputs a retrieval result to the incentive provision section 111.

In a case where any record corresponding to the applicable key is present, the incentive provision section 111 determines that an available incentive is present. The processing in step S704 has been described above.

In a case where no applicable incentive is present, the incentive provision section 111 ends the analysis result reception processing.

In a case where an applicable incentive is present, the incentive provision section 111 executes incentive determination processing (step S705). The processing in step S705 is similar to the processing in step S204. However, the processing in step S705 differs from the processing in step S204 in processing executed by a program invoked by the incentive provision section 111.

The program having received the activation instruction outputs, to the access control section 113, the service retrieval request including the applicable key.

The access control section 113 retrieves a record including the applicable key 802 in which the applicable key included in the service retrieval request is stored. The access control section 113 outputs the retrieved record to the program.

The program uses the received record to generate additional information useful for health improvement of at least one of the parent and the child. The program outputs the generated additional information to the incentive provision section 111.

The incentive provision section 111 transmits the additional information to the identified mobile terminal 101 as a second incentive (step S706). Subsequently, the incentive provision section 111 ends the analysis result reception processing.

Now, an example of the additional information generated by the program invoked by the incentive provision section 111 will be described.

(1) In a case where the parent-and-child health study assistance system 100 receives analysis result data indicating a lack of exercise, the program generates additional information used to overcome the lack of exercise. For example, processing as described below is executed to generate additional information.

The program utilizes the system 102 providing service associated with the applicable key to acquire map information regarding the residential area of the user and surrounding areas and information of events in and around the residential area of the user. The event information is information regarding events related to the region, such as a construction project and a crop-dusting schedule. The map information can be acquired from a service provider providing maps. The event information such as a construction project and a crop-dusting schedule can be acquired from the local government, a construction company, an agricultural association, and the like.

The program generates a route for walking on the basis of the map information and the event information, and generates additional information in which the generated route is stored.

(2) In a case where the parent-and-child health study assistance system 100 receives analysis result data indicating a deficiency of a nutrient, the program generates additional information indicating products for supplementing the nutrient and restaurants providing meals for supplementing the nutrient. For example, processing as described below is executed to generate additional information.

The program generates additional information indicating products and restaurants associated with the applicable key. At this time, the program may generate map information regarding the residential area of the user and surrounding areas and generate, in the map, additional information indicating the sales locations of products and the locations of restaurants, and the contents of the products and menus.

(3) In a case where the parent-and-child health study assistance system 100 receives analysis result data indicating a deficiency of a nutrient, the program generates additional information indicating menus or improvement methods for supplementing the nutrient. For example, processing as described below is executed to generate additional information.

The program transmits a provision request including the deficient nutrient to the system 102 providing the service associated with the applicable key. The program receives information regarding the menus or the improvement methods from the system 102 and generates additional information indicating the menus or the improvement methods.

According to the analysis result, the parent-and-child health study assistance system 100 provides, as the second incentive, additional information useful for health improvement of the parent and the child. This allows the use by new users and the continued use by existing users to be promoted.

The parent-and-child health study assistance system 100 can provide health study data or the like corresponding to the type of the service of the service provider. This enables the service provider to carry out research, product development, service creation, and the like. Additionally, by registering a service in the parent-and-child health study assistance system 100, the service provider can provide the service to targeted users or capture opportunities to provide the service. As described above, the parent-and-child health study assistance system 100 is also advantageous to service providers, and thus the number of services available can be expected to be increased via the parent-and-child health study assistance system 100. An increasing number of services available allows the use by new users and the continued use by existing users to be promoted.

Note that the present invention is not limited to the above-described embodiments and includes various modified examples. Additionally, for example, the embodiments have been described in detail in terms of configuration in order to describe the present invention in an easy-to-understand manner and the present invention is not necessarily restricted to embodiments that include all configurations described hereinabove. Additionally, a part of the configuration of each of the embodiments may be added to another configuration, omitted, or replaced with another configuration.

Additionally, for example, by designing with integrated circuits, a part or all of each of the above-described configurations, functions, processing sections, processing means, and the like may be implemented by hardware. In addition, the present invention can be implemented by a program code of software realizing the functions of the embodiments. In this case, a computer is provided with a storage medium in which the program code is recorded, and a processor provided in the computer reads out the program code stored in the storage medium. In this case, the program code itself read out from the storage medium implements the functions of the embodiments described above, and the program code itself and the storage medium storing the program code constitute the present invention. The storage medium for delivering such a program code includes a flexible disk, a CD-ROM, a DVD-ROM, a hard disk, an SSD (Solid State Drive), an optical disc, a magneto-optical disc, a CD-R, a magnetic tape, a nonvolatile memory card, a ROM, and the like.

Additionally, the program code realizing the functions described in the embodiments can be implemented by, for example, a wide variety of programs or script languages such as an assembler, C/C++, perl, Shell, PHP, Python, and Java.

Furthermore, the program code of software implementing the functions of the embodiments may be distributed via a network and stored in a storage means of a computer such as a hard disk or a memory or in a storage medium such as a CD-RW or a CD-R, and a processor provided in the computer may read out and execute the program code stored in the storage means or the storage medium.

In the above-described embodiments, control lines and information lines indicate those which are considered necessary for description and do not necessarily indicate all the control lines and information lines of the product. All the components may be connected together.

1. A parent-and-child health study assistance system comprising:
a plurality of terminals held by a user; and
at least one computer connected to the plurality of terminals, wherein
the at least one computer includes an arithmetic apparatus and a storage apparatus connected to the arithmetic apparatus,
the parent-and-child health study assistance system includes:
a data management section managing health study data used for a health study of at least one of a parent and a child of a family that includes the user as a family member;
a level calculation section calculating a contribution level for the health study of the user based on input of the health study data;
an incentive provision section providing a first incentive as a profit corresponding to the contribution level, to the user via the terminal in conjunction with input of the health study data;
an access control section managing an access authority to access the health study data; and
an analysis result provision section providing an analysis result of an analysis process using the health study data, and
the access control section determines, on a basis of an attribute of a service provider, the access authority granted to the provider.

2. The parent-and-child health study assistance system according to claim 1, wherein
the incentive provision section generates, on a basis of the analysis result, additional information useful for health improvement of one of the parent and the child, and provides the additional information as a second incentive.

3. The parent-and-child health study assistance system according to claim 2, wherein
in a case where the analysis result includes information pointing out a lack of exercise,
the incentive provision section
identifies a residential area of the user using the terminal that have transmitted the health study data used to generate the analysis result, and identifies an event in the residential area, and
generates the additional information presenting a walking route generated on a basis of the residential area and the event.

4. The parent-and-child health study assistance system according to claim 2, wherein
in a case where the analysis result includes information pointing out a deficiency of a nutrient, the incentive provision section generates the additional information presenting the service for solving the deficiency of the nutrient.

5. The parent-and-child health study assistance system according to claim 4, wherein
the incentive provision section
identifies a location where a service for solving the nutrient deficiency is available at the residential area of the user using the terminal that have transmitted the health study data used to generate the analysis result, and
generates the additional information presenting the service for solving the nutrient deficiency and the identified location.

6. The parent-and-child health study assistance system according to claim 1, wherein
the analysis result provision section provides the analysis result including an estimated levels of vitamin D in the body calculated on a basis of the health study data related to going out.

7. The parent-and-child health study assistance system according to claim 1, wherein
the level calculation section calculates the contribution level on a basis of the number of inputs of the health study data.

8. The parent-and-child health study assistance system according to claim 1, wherein
the level calculation section calculates the contribution level on a basis of a content of the health study data.

9. A parent-and-child health study assistance apparatus to which a plurality of terminals held by a user are connected, the parent-and-child health study assistance apparatus comprising:
a data management section managing health study data used for a health study of at least one of a parent and a child of a family in which the user is a family member;
a level calculation section calculating a contribution level for the health study of the user, by inputting the health study data;
an incentive provision section providing a first incentive, which is used as a profit corresponding to the contribution level, to the user via the terminal in conjunction with input of the health study data;
an access control section managing an access authority to access the health study data; and
an analysis result provision section providing an analysis result of analysis processing using the health study data, wherein
the access control section determines, on a basis of an attribute of a service provider, the access authority granted to the provider.

10. The parent-and-child health study assistance apparatus according to claim 9, wherein
the incentive provision section generates, on a basis of the analysis result, additional information useful for health improvement of one of the parent and the child, and provides the additional information as a second incentive.

11. A data processing method executed by a parent-and-child health study assistance system including a plurality of terminals held by a user, and at least one computer connected to the plurality of terminals,
the at least one computer including an arithmetic apparatus and a storage apparatus connected to the arithmetic apparatus,
the parent-and-child health study assistance system managing health study data used for a health study of at least one of a parent and a child of a family that includes the user as a family member,
the data processing method comprising:
a first step, wherein the at least one computer calculates a contribution level for the health study of the user in conjunction with input of the health study data;
a second step, wherein the at least one computer provides a first incentive to the user as a profit corresponding to the contribution level;
a third step, wherein the at least one computer determines an access authority of the provider to access the health study data on a basis of the attribute, in a case of receiving a registration request including an attribute of a service provider; and
a fourth step, wherein the at least one computer provides the user with an analysis result of an analysis process using the health study data.

12. The data processing method according to claim 11, wherein
the fourth step includes,
a step in which the at least one computer generates additional information useful for health improvement of one of the parent and the child, on a basis of the analysis result; and
a step in which the at least one computer transmits the additional information to the terminal as a second incentive.
